(19) United States
(12) Patent Application Publication
(10) Pub. No.: US 2013/0216630 A1
(43) Pub. Date: Aug. 22, 2013

(54) BIOMATERIALS FOR USE IN METHODS OF BONE REPLACEMENT THERAPY

(71) Applicants: Medical Research Council of South Africa, (US); University of Witwatersrand, Johannesburg, (US)

(72) Inventors: Ugo Ripamonti, Parktown (ZA); Carlo Ferretti, Johannesburg (ZA)

(73) Assignees: UNIVERSITY OF WITWATERSRAND, JOHANNESBURG, Johannesburg (ZA); MEDICAL RESEARCH COUNCIL OF SOUTH AFRICA, Capetown (ZA)

(21) Appl. No.: 13/845,253

(22) Filed: Mar. 18, 2013

Related U.S. Application Data
(63) Continuation of application No. 13/183,903, filed on Jul. 15, 2011.

(30) Foreign Application Priority Data
Jul. 15, 2010 (ZA) 2010/03648

(51) Int. Cl. A61K 31/675; A61K 33/10; A61K 38/18; A61K 33/42

(52) U.S. Cl. CPC A61K 31/675; A61K 33/42; A61K 33/10; A61K 38/1875; A61K 38/1841
USPC 424/602; 548/112; 424/687; 514/16.7; 514/8.8; 514/94; 514/8.9

(57) ABSTRACT

This invention relates to biomaterials, said biomaterials for use in methods to control and/or induce bone growth. Particularly, the invention relates to macroporous calcium phosphate biomaterials pre-loaded with certain amounts of osteoclastic activity inhibitors for use in methods to control and/or induce bone growth in primates.

### PRIORITY CLAIM

This application claims priority under 35 U.S.C. §119 to South African Provisional Application Serial Number 2010/03648, of Jul. 15, 2010, the disclosures of which are expressly incorporated by reference herein.

### FIELD OF THE INVENTION

This invention relates to biomaterials for use in methods to control and/or induce bone growth particularly, it relates to macroporous calcium phosphate biomaterials for use in methods of bone replacement therapy, furthermore particularly it relates to macroporous calcium phosphate biomaterials pre-loaded with certain amounts of osteoclastic activity inhibitors for use in methods of bone replacement therapy in primates.

### BACKGROUND TO THE INVENTION

Recent years have shown a surge in research output in the biomaterials field, said research particularly considering the interplay between the biomaterials field and associated biological research areas such as tissue engineering, regenerative medicine and stem cell research [(1) D. Williams, Biomaterials, 32, 1-2(2010); the entirety of which is incorporated by reference herein]. Current and future trends in biomaterials research will revolve around the functionality and bioactivity of in vivo implanted biomaterials as biomimetic matrices and their ability to interact with specific molecular and tissue biology phenomena in order to induce regenerative responses as inductive biomaterials [(2) Ripamonti, U., Advanced Bioactive biometric matrices induce bone formation by auto-induction, 11^{th} ICFPAM Conference Symposium 8: Biomaterials Africa 2011; the entirety of which is incorporated by reference herein].

Inductive biomaterials per se and without the exogenous application of soluble molecular signals, trigger the ripple-like cascade of pattern formation and tissue induction that initiate the generation of morphogenesis [(2)]. The basic tissue engineering paradigm is tissue induction and morphogenesis by combinatorial molecular protocols whereby soluble molecular signals are recombined with insoluble signals or substrata acting as tridimensional constructs for the initiation of de novo tissue induction and morphogenesis [(3) U. Ripamonti, J.Cell.Mol.Med., 12, (6B), 2953-2972 (2009); the entirety of which is incorporated by reference herein]. The paradigm has been modified by the language of geometry [(2), (4) Ripamonti, U. Inductive bone matrix and porous hydroxyapatite composites in rodents and nonhuman primates. Handbook of bioactive ceramics, vol II. CRC Press; 1990. 245-53.; the entirety of which is incorporated by reference herein] in that the lacunae, pits and concavities that are cut by osteoclastogenesis within the biomimetic matrices are the driving morphogenetic cues that set in motion the induction of bone formation [(4)].

It is known that heterotopic extraskeletal implantation of macroporous calcium phosphate-based biomaterials into the rectus abdominis muscle of the non-human primate Papio ursinus results in the ‘spontaneous’ induction of bone formation and without the exogenous application of the osteogenic proteins of the transforming growth factor-β supergene family, the bone morphogenetic/osteogenic proteins (BMPs/OPs) or, uniquely in primates, the mammalian transforming growth factor-β (TGF-β) proteins. It has been shown that several naturally-derived and/or synthesized calcium/phosphate macroporous constructs induce the differentiation of bone when implanted intramuscularly in Papio ursinus. However, control of the bone growth induction process and control of the bone growth is essential in order to provide for growth which is selectively tailored in order to provide for a specific pre-determined structural entity. Consequently, there is a need for novel biomaterials for use in novel methods to control induction and growth of bone formation.

### OBJECT OF THE INVENTION

It is an object of the invention to provide biomaterials for use in methods of bone replacement therapy which will, at least partially, overcome the abovementioned problems.

### SUMMARY OF THE INVENTION

According to a first aspect of the invention there is provided a biomaterial comprising: a solid porous composition, preferably the solid porous composition being a calcium carbonate containing macroporous composition, further preferably a calcium phosphate containing composition; and an amount of zoledronate or a zoledronate containing compound, in use, the amount of zoledronate or the zoledronate containing compound is applied to the solid composition.

There is provided that the biomaterial be adapted to form a solid construct, preferably a solid matrix.

There is provided for the zoledronate containing compound to be a biphosphonate zoledronate containing compound.

In one exemplary embodiment of the invention the solid porous composition is a coral-derived calcium carbonate-based macroporous composition, preferably the coral-derived calcium carbonate-based macroporous composition having a hydrothermal conversion to hydroxyapatite of about 7%.

There is provided for the biomaterial to further include a naturally-derived or recombinant human bone morphogenetic/osteogenic protein, preferably osteogenic protein-1 (OP-1).

There is further provided for the biomaterial to further include a transforming growth factor-β, typically TGF-β₃.

There is further provided in a preferred embodiment of the invention for the biomaterial to be shaped and dimensioned into a predetermined form, preferably the predetermined form being further deformable, such that in use, the biomaterial is placed into a desired position and is further deformed to secure it in the desired position, preferably the desired position being a site located within a body of a mammal, specifically the mammal being a primate.

There is provided that the biomaterial further comprises a holding means, the holding means in use facilitating holding the biomaterial to a desired position, preferably the desired position being a site located within a body of a mammal, specifically the mammal being a primate.

There is further provided for the biomaterial to further comprise at least one pharmaceutical composition, typically including an excipient and/or an adjuvant.

According to a second aspect of the invention there is provided a method of bone replacement therapy for animals and/or humans having bone degeneration and/or bone deformation and/or bone loss, the method comprising: inserting a solid porous composition into a body of a mammal, preferably the solid porous composition being a calcium carbonate containing composition, further more preferably a calcium phosphate containing composition; and adding zoledronate or a zoledronate containing compound to the solid porous composition, in use, the solid porous composition stimulating bone growth and the zoledronate inhibiting bone growth such that bone the rate of bone growth is modulated.

There is provided that the solid porous composition be adapted to form a solid construct, particularly a solid matrix.

In an exemplary embodiment of the invention the solid porous composition is a coral-derived calcium carbonate-based macroporous composition, specifically the coral-derived calcium carbonate-based macroporous composition having a hydrothermal conversion to hydroxyapatite of about 7%.

There is provided for the solid porous composition to further include a naturally-derived or recombinant human bone morphogenetic/osteogenic protein, typically osteogenic protein-1 (OP-1).

There is further provided for the solid porous composition to further include a transforming growth factor-ft typically TGF-β₃.

There is further provided in an exemplary embodiment of the invention, a method of bone replacement therapy for animals and/or humans having bone degeneration and/or bone deformation and/or bone loss, to include the step of shaping and/or dimensioning the solid porous composition into a predetermined form, such as the predetermined form being further deformable, such that in use, the solid porous composition is inserted at a desired site inside a body of a mammal, specifically a primate, and is further deformed to secure it at the desired site.

There is further provided for the method to include the step of securing the solid porous composition to the desired site via a holding means.

There is further provided for the method to include the step of adding a pharmaceutical composition, preferably including an excipient and/or adjuvant, to the solid porous composition.

According to an third aspect of the invention there is provided a method of bone replacement therapy for animals and/or humans having bone degeneration and/or bone deformation and/or bone loss, the method comprising: inserting a biomaterial as described in the first aspect of the invention into a body of a mammal, in use, the solid porous composition stimulating bone growth and the zoledronate inhibiting bone growth such that the biomaterial modulates the rate of bone growth in an animal or human into which the biomaterial has been inserted.

According to a fourth aspect of the invention, there is provided a method of controlling induction of bone formation and/or bone growth, the method comprising: contacting a solid porous composition, more preferably a calcium carbonate containing composition, further more specifically a calcium phosphate containing composition, with a biological sample, in particular the sample being a tissue culture, further particularly the tissue culture being a mammalian tissue culture; and adding zoledronate or a zoledronate containing compound to the solid porous composition in contact with the biological sample.

According to a fifth aspect of the invention, there is provided a method of controlling induction of bone formation and/or bone growth, the method comprising: inserting a solid porous composition into a body of a mammal, specifically the solid porous composition being a calcium carbonate containing composition, further more specifically a calcium phosphate containing composition; and adding zoledronate or a zoledronate containing compound to the solid porous composition.

There is provided that the solid porous composition be adapted to form a solid construct, such a solid matrix.

In an exemplary embodiment of the invention the solid porous composition is a coral-derived calcium carbonate-based macroporous composition, specifically the coral-derived calcium carbonate-based macroporous composition having a hydrothermal conversion to hydroxyapatite of about 7%.

There is provided for the solid porous composition to further include a naturally-derived or recombinant human bone morphogenetic/osteogenic protein, typically osteogenic protein-1 (OP-1).

There is further provided for the solid porous composition to further include a transforming growth factor-β typically TGF-β₃.

There is further provided in an exemplary embodiment of the invention, a method of controlling induction of bone formation and/or bone growth, to include the step of shaping and/or dimensioning the solid composition into a predetermined form, specifically the predetermined form being further deformable, such that in use, the solid porous composition is inserted at a desired site inside a body of a mammal, particularly a primate, and is further deformed to secure it at the desired site.

There is further provided for the method to include the step of securing the solid porous composition to the desired site via a holding means.

There is further provided for the method to include the step of adding a pharmaceutical composition, specifically including an excipient and/or an adjuvant, to the solid porous composition.

According to a sixth aspect of the invention, there is provided a method of controlling induction of bone formation and/or bone growth, the method comprising: contacting a biomaterial as described in the first aspect of the invention with a biological sample, specifically the sample being a tissue culture, in particular the tissue culture being a mammalian tissue culture.

According to a seventh aspect of the invention, there is provided a method of controlling induction of bone formation and/or bone growth, the method comprising: inserting a biomaterial as described in the first aspect of the invention into a body of a mammal.

According to an eighth aspect of the invention, there is provided a use of a biomaterial as described in the first aspect of the invention in a method for controlling and/or inducing bone growth, the use comprising inserting said biomaterial into a body of a mammal, preferably a primate, the solid porous composition stimulating bone growth and the zoledronate inhibiting bone growth such that the biomaterial controls and/or induces bone growth in an animal or human into which the biomaterial has been inserted.

According to a ninth aspect of the invention there is provided a use of zolendronate or a zolendronate containing compound in a method for controlling and/or inducing bone growth, the use comprising contacting the zolendronate or the zolendronate containing compound with a solid porous composition, which solid porous composition is inserted into a body of a mammal, specifically a primate, the solid porous composition stimulating bone growth and the zoledronate inhibiting bone growth such that the biomaterial controls and/or induces bone growth in an animal or human into which the biomaterial has been inserted.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The above mentioned and other features and objects of this invention, and the manner of attaining them, will become more apparent and the invention itself will be better understood by reference to the following description of an embodiment of the invention taken into conjunction with the accompanying drawings, wherein:

FIG. 1 shows a series of samples (A-F) highlighting the lack of bone induction and/or bone growth by coral-derived 7% HA-CC pre-treated with zoledronate 90 days after implantation in the rectus abdominis muscles of adult Papio ursinus;

FIG. 1A sample shows a low power view of a first sample;

FIG. 1B sample shows a magnified view of the area highlighted in 1A, note the lack of bone differentiation in the zoledronate treated specimen;

FIG. 1C sample shows another low power view of a second sample, the arrow indicating newly formed bone confined to the periphery of the treated sample;

FIG. 1D sample shows a magnified view of the area highlighted by an arrow in 1C, showing minimal bone formation in a zoledronate treated sample;

FIG. 1E sample shows a high powered magnification of a third sample; and

FIG. 1F sample shows a high powered magnification of a fourth sample, again with minimal induction of bone formation at the periphery of the specimen only.

Corresponding reference characters indicate corresponding parts throughout the several views. Although the drawings represent embodiments of the present invention, the drawings are not necessarily to scale and certain features may be exaggerated in order to better illustrate and explain the present invention. The exemplifications set our herein illustrate embodiments of the invention, in several forms, and such exemplifications are not to be construed as limiting the scope if the invention in any manner.

### DETAILED DESCRIPTION OF EMBODIMENTS OF THE INVENTION

The embodiments disclosed below are not intended to be exhaustive or limit the invention to the precise forms disclosed in the following detailed description. Rather, the embodiment is chosen and described so that others skilled in the art may utilize its teaching. It has been shown that osteoclastic activity is prominent along macroporous constructs inserted into a non-human primate. It has also been observed that osteoclasts, activated by the implantation of the macroporous biomaterials, attach to the implanted biomaterial and topographically modify the surface geometry of the construct surface. Modifications are in the range of a thousand microns or less in the form of lacunae, pits and concavities cut by osteoclastogenesis [(1)-(4)].

Topographical modification of the implanted constructs may be of the utmost importance for the differentiation of myoblastic/myoendothelial and/or perivascular/endothelial stem cells into osteoblastic-like cells. In order to study the effect of the osteoclastic activity on cell differentiation and the induction of bone formation, samples of solid porous construct compositions, typically macroporous hydroxyapatites were pre-loaded with 0.24 mg of the osteoclastic inhibitor biphosphonate zoledronate (Zometa®). Samples with and without zoledronate were then implanted in the rectus abdominis muscle of Papio ursinus. Histological evaluation of histological sections cut from the implanted specimens harvested 90 days after implantation showed that untreated macroporous constructs did induce the differentiation of bone within the macroporous spaces, and that specimens pre-loaded with 0.24 mg of the biphosphonate zoledronate either did not induce the differentiation of bone at all, or the induction was considerably inhibited.

An aspect of the invention, a method of bone replacement therapy for animals and/or humans having bone degeneration and/or bone deformation and/or bone loss, is described by simply pre-loading a macroporous construct with 0.24 mg of the osteoclastic inhibitor biphosphonate zoledronate (Zometa®), and inserting said pre-loaded construct into a mammal, preferably a primate.

The lack of osteoclastic activity results in the lack of topographical macroporous surface modifications in the form of lacunae, pits and concavities; the lack of topographical macroporous modifications results in the lack of stem cell differentiation into osteoblastic-like cells; the lack of osteoblastic-like cells result in the lack of BMPs/OPs expression, synthesis and secretion; the lack of BMPs/OPs results in the lack of bone differentiation and/or bone growth.

It is to be understood that treatment of a solid porous construct with hOP-1, hTGF-β3, and bisphosphonate zoledronate Zometa® can be tailored to provide for a biomaterial which will result in a desired rate of bone induction and/or bone growth in a mammal, preferably a primate. This is owing to the fact that hOP-1 and hTGF-β3 typically induces bone induction and/or bone growth but zoledronate compounds inhibit bone induction and/or bone growth. It is to be understood that the solid porous constructs can be pre-treated before implantation or that treatment may occur post-implantation. By employing the methods described herein it becomes possible to control the rate and extent of bone induction and/or bone growth.

### Examples

The invention will be described with reference to the below non-limiting examples.

A biomaterial to act, in use, as a platform for bone growth induction and/or bone growth was prepared. Said solid porous composition in the form of macroporous hydroxyapatite replicas of the calcium carbonate exoskeletal microstructure of the coral genus Goniopora was prepared by hydrothermal chemical exchange with phosphate [(5) Ripamonti, U. J Bone Joint Surg Am 1991; 73:692-703; (6) Ripamonti U, Richter P W, Nilen R W N, Renton L., J Cell Mol Med 2008; 12:1029-48., (7) Shors EC, Orthop Clin North Am 1999:30:599-613; (8) Ripamonti U, van den Heever B, van Wyk J., Matrix 1993;13:491-502; (9) Ripamonti, R. M. Klar, L. F. Renton, C Ferretti., Biomaterials, 31, 6400-6410 (2010). The entirety of which are incorporated by reference herein]. The preparation of the solid porous composition resulted in solid calcium carbonate (CC) constructs with 7% hydroxyapatite (HA) designated as 7% hydroxyapatite/calcium carbonate (7% HA/CC) constructs [(7), (8)]. The solid porous composition was processed into implantable constructs or rods for heterotopic implantation in Papio ursinus. The rods were about 7 mm in diameter and about 20 mm length. The solid components of the solid porous composition averaged about 130 μm diameter and their interconnection about 220 μm; the average porosity was about 600 μm and their interconnections average about 260μm in diameter [(5)-(9)]. The constructs are optimal biomimetic substrata for cell attachment, proliferation and differentiation, acting as non-immunogenic carriers for the biological activity of the osteogenic proteins of the TGF-β supergene family [(5)-(9)]. The macroporous 7% HA/CC constructs were coated with bisphosphonate zoledronate Zometa® solution to form a biomaterial. The coated constructs were implanted into the rectus abdominis of an adult Papio ursinus.

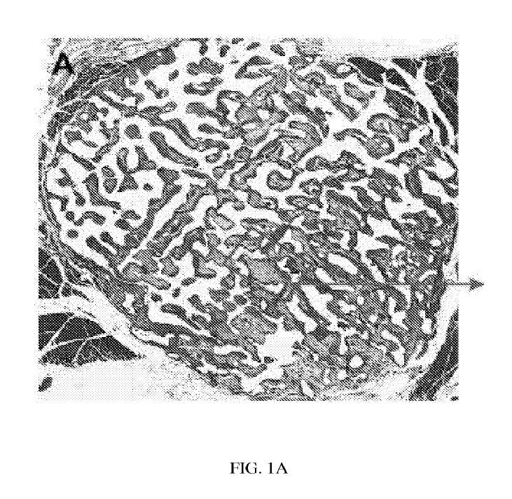

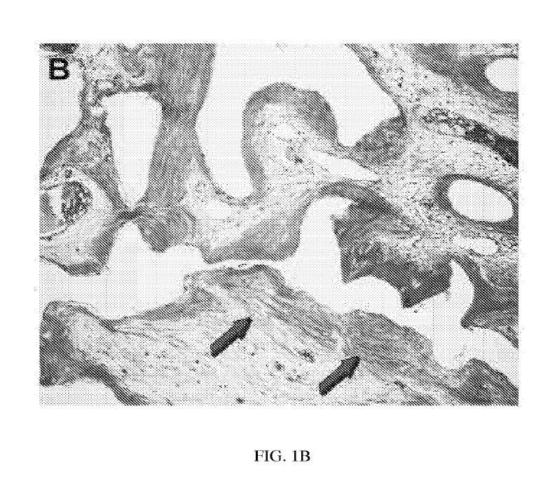

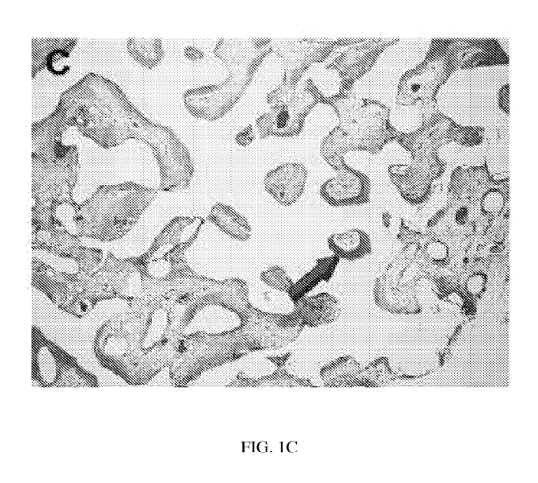

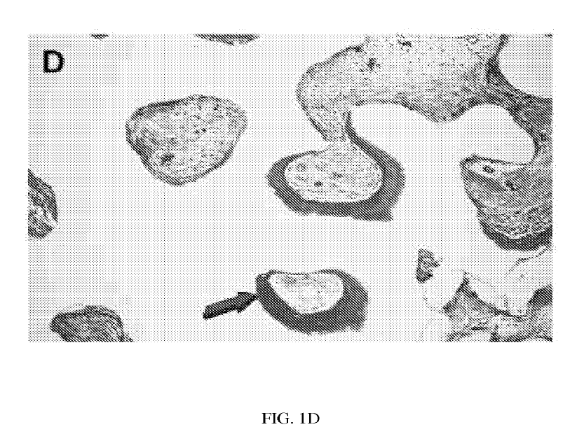

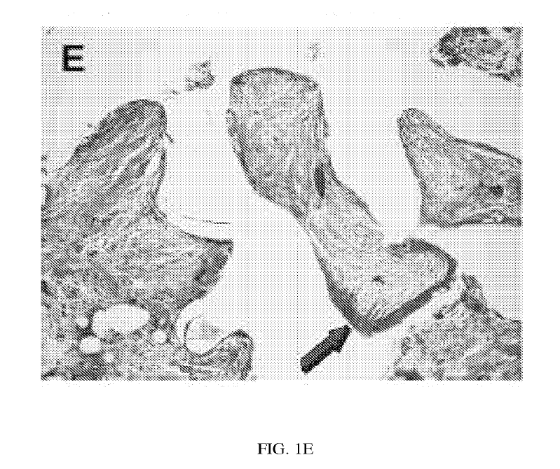

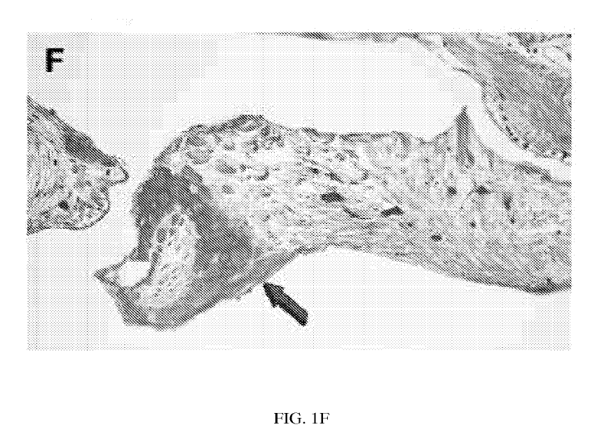

At harvest, 90 days after implantation, zoledronate pre-treated macroporous constructs showed fibrovascular invasion and collagenous condensations across the macroporous spaces tightly attached to the implanted substratum as shown in FIGS. 1A and B. In two specimens, the spontaneous induction of bone formation was altogether absent as shown in FIGS. 1A and B. Morphological analyses of the remaining specimens showed that minor amounts of bone had formed by induction at the periphery of the implanted pre-treated macroporous scaffolds. The induction of bone, albeit limited at the periphery as is evident in FIGS. 1C to F, may be the result of incomplete diffusion of the zoledronate throughout the entirety of the macroporous spaces.

qTR-PCR analyses of the harvested biomaterial constructs showed a dramatic reduction of osteogenic protein-1 (OP-1) gene expression in specimens pre-treated with 0.24 mg zoledronate. It is important to note that in specimens with bone formation by induction there is always expression of the OP-1 gene, i.e. there is a direct correlation between the induction of bone and the expression of the OP-1 gene.

Significantly, macroporous constructs pre-treated with 0.24 mg zoledronate (the biomaterial of this invention) showed very limited bone formation and two specimens lacked the initiation of bone formation altogether. Osteoclastic post-implantation modifications of the implanted macroporous substrata are thus critical for the induction of macro- and micropatterned topographies highly suitable for the differentiation of resident stem cells into osteoblastic-like cells expressing the soluble osteogenic molecular signals of the TGF-β supergene family.

Further details describing the invention are described in Biomaterials, 31, 6400-6410, (2010) by U. Ripamonti, R. M. Klar, L. F. Renton and C. Ferretti, the entirety of which is incorporated by reference herein.

While this invention has been described as having an exemplary design, the present invention may be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains. The following Appendices provide examples of aspects of the present invention, which may be altered and adapted in various forms.

1. A biomaterial comprising a solid porous composition containing zoledronate.

2. The biomaterial according to claim 1, wherein the solid porous composition comprises at least one of the group: a calcium carbonate containing composition, a calcium phosphate containing composition and coral-derived calcium carbonate-based macroporous composition having a hydrothermal conversion to hydroxyapatite of about 7%.

3. The biomaterial according to claims 1, wherein the zoledronate is in the form of a zoledronate containing composition.

4. The biomaterial according to claim 3, wherein the biphosphonate zoledronate containing composition optionally includes at least another pharmaceutically active compound and/or an excipient and/or an adjuvant.

5. The biomaterial according to claim 1, wherein the biomaterial further includes a naturally-derived human bone morphogenetic/osteogenic protein.

6. The biomaterial according to claim 5, wherein the naturally-derived human bone morphogenetic/osteogenic protein is osteogenic protein-1 (OP-1).

7. The biomaterial according to claim 1, wherein the biomaterial further includes a recombinant human bone morphogenetic/osteogenic protein.

8. The biomaterial according to claim 7, wherein the recombinant human bone morphogenetic/osteogentic protein is osteogenic protein-1 (OP-1).

9. The biomaterial according to claim 1, wherein the biomaterial further includes a transforming growth factor-β.

10. The biomaterial according to claim 1, wherein the transforming growth factor-β is TGF-β₃.

11. The biomaterial according to claim 1, wherein the biomaterial is shaped and dimensioned into a predetermined from and is further deformable, such that in use, the biomaterial is placed into a body of a mammal, preferably the mammal being a primate.

12. A method of bone replacement therapy for animals and/or humans having bone degeneration and/or bone deformation and/or bone loss, the method comprising:
inserting a solid porous composition into a body of a mammal; and
adding zoledronate to the biomaterial, in use, the solid porous composition stimulates bone growth and the zoledronate inhibits bone growth such that the rate of bone growth is modulated.

13. The method of claim 12, wherein the solid porous composition comprises at least one of the group: a calcium carbonate containing composition, a calcium phosphate containing composition and coral-derived calcium carbonate-based macroporous composition having a hydrothermal conversion to hydroxypatite of about 7.

14. The method according to claim 12, wherein the zoledronate is in the form of a zoledronate containing composition.

15. The method according to claim 14, wherein the zoledronate composition optionally includes at least another pharmaceutically active compound and/or an excipient and/or an adjuvant.

16. The method according to claim 12, wherein the solid porous composition further includes a naturally-derived or recombinant human bone morphogenetic/osteogenic protein and/or transforming growth factor-β, preferably the naturally-derived or recombinant human bone morphogenetic/osteogenic protein is osteogenic protein-1 (OP-1), and preferably the transforming growth factor-β is TGF-β₃.

17. The method according to claim 12, wherein the solid porous composition is shaped and dimensioned into a predetermined form and is further deformable, such that in use, solid porous composition is placed into a body of a mammal/

18. The method according to claim 17, wherein the mammal is a primate.

19. The method according to claim 17, further comprising the step of securing the solid porous composition to a mammal via a holding means.

20. A method of bone replacement therapy for animals and/or humans having bone degeneration and/or bone deformation and/or bone loss, the method comprising:
inserting the biomaterial comprising a solid porous composition containing zoledronate into a body of a mammal, in use, the solid porous composition stimulating bone growth and the zoledronate inhibiting bone growth such that the biomaterial modulates the rate of bone growth.
